(19) United States
(12) Patent Application Publication
(10) Pub. No.: US 2004/0265385 A1
(43) Pub. Date: Dec. 30, 2004

(54) Porous biostructure partially occupied by interpenetrant and method for making same

(75) Inventor: Thomas George West, Lawrenceville, NJ (US)

Correspondence Address: HUNTON & WILLIAMS LLP, INTELLECTUAL PROPERTY DEPARTMENT, 1900 K STREET, N.W., SUITE 1200, WASHINGTON, DC, 20006-1109, US

(73) Assignee: Therics, Inc., Princeton, NJ

(21) Appl. No.: 10/837,913

(22) Filed: May 3, 2004

Related U.S. Application Data
(63) Continuation-in-part of application No. 10/122,129, filed on Apr. 12, 2002.
(60) Provisional application No. 60/467,474, filed on May 1, 2003.
(60) Provisional application No. 60/486,404, filed on Jul. 11, 2003.
(60) Provisional application No. 60/488,362, filed on Jul. 17, 2003.
(60) Provisional application No. 60/283,564, filed on Apr. 12, 2001.

(51) Int. Cl. A61K 9/14

(52) U.S. Cl. 424/484

(57) ABSTRACT

A biostructure including a porous matrix, the interstitial pores of the matrix selectively infused with an interpenetrant such that portions of the matrix remain uninfused. The biostructure may include a ceramic matrix and a polymer interpenetrant. The biostructure may be an implantable bone substitute including a bone repair device, a cranioplasty device, a burr hole cover or cap, a mandibular repair device, other craniofacial repair device, an alveolar ridge augmentation, bone void filler, a spinal fusion or other spinal repair device, or other substitute for either a portion of a bone or an entire bone. The biostructure, or its corresponding matrix, may have dimensions which may be customized for a particular patient and which may be based on medical imaging data and may further include geometric features not present in the medical imaging data. The biostructure may be used in culturing cells outside the body of a patient.

### CROSS-REFERENCE TO RELATED APPLICATIONS

This application claims the benefit of Provisional Application filed May 1, 2003, titled “SELECTIVE INFUSION,” application No. 60/467,474, and Provisional Application filed Jul. 11, 2003, titled “BIOMECHANICAL TESTING OF OSTEOCONDUCTIVE DISKS FOR CRANIOPLASTY IN AN OVINE MODEL,” application No. 60/486,404, and Provisional Application filed Jul. 17, 2003, titled “POROUS BIOSTRUCTURE PARTIALLY OCCUPIED BY INTERPENETRANT AND METHOD FOR MAKING SAME,” application No. 60/488,362, and; each of which is incorporated herein by reference in its entirety.

### BACKGROUND OF THE INVENTION

1. Field of the Invention

This invention relates generally to biostructures such as biostructures conducive to the ingrowth, repair and healing of natural bone and tissue, and methods of making the same.

2. Description of the Related Art

Porous ceramics, notably the various calcium phosphates (hydroxyapatite, tricalcium phosphate, etc.), as well as certain other materials, are known to be useful as bone substitute materials. Factors that influence the suitability of materials for use as bone substitutes include their ability to support or encourage the ingrowth of natural bone and tissue, and their mechanical properties, such as strength and fracture toughness. The importance of the mechanical properties varies depending on the specific location and loading of a bone.

In general, porosity is known to support or encourage the ingrowth of natural bone and tissue. However, entirely porous biostructures made from ceramics have been known to suffer from the inherent brittleness of the materials themselves, resulting in a tendency of the biostructure to fracture easily under mechanical loading. Consequently, a number of approaches have been used to toughen these materials so that they can survive handling, manipulation, implantation and loading during use prior to bone ingrowth. One of the most common techniques has been to infiltrate the porous structure with another material, such as a polymer, to occupy the void space and impart additional strength and toughness to the biostructure.

In order to provide both porosity and strength in a single biostructure, biostructures have been designed which have included an outer porous layer, which has allowed bone to contact and integrate directly with the surface of the biostructure, together with an interior which has been made more solid for purposes of mechanical strength. Giordano et al. (U.S. Pat. No. 6,605,293) has described a technique for making such a biostructure in which a porous preform has been manufactured, a fugitive material has been applied to outer surface regions of the preform, infusion media have been infiltrated into the core to form an interpenetrating phase composite in the core, and finally the fugitive material has been removed to reveal the outer porous region. While this technique has achieved interpenetrant-free porous regions, it has been able to achieve such regions essentially only along portions of the overall exterior surface of the biostructures. Interpenetrant-free regions at more arbitrary locations have not been achieved.

For example, interpenetrant-free regions at the boundaries of possible internal channels, whose cross-sectional dimensions may be of the order of hundreds of microns, would be desirable but have not been achieved. Also, the method does involve process steps associated with applying and then removing the fugitive material. Another feature of Giordano is that regions receiving infiltration (i.e., are not blocked by the fugitive material) have been substantially completely infiltrated with the interpenetrant, resulting in significant discontinuity at the boundary between the two regions. This discontinuity may be undesirable for reasons of mechanical stress concentration, especially if the polymer is nonresorbable (i.e., persists indefinitely in the body of the patient). There has been no disclosure about partially filled pores in what Giordano describes as the inner core of the prosthesis.

In other literature, it has been found that, in order to achieve significant bone ingrowth for a biostructure that has a pore size distribution, it is advantageous to concentrate infiltration on small pores of a biostructure while leaving some large pores relatively unfilled. White et al. (U.S. Pat. No. 6,376,573 B1) has described a technique that has allowed infiltration of the micropores (below 1 micrometer in size) of a porous preform while leaving only a coating on what he refers to as the macropores (100-1000 micrometers in size). The preferred method involved gradually dipping a preheated preform into a preheated liquid infiltrant medium, allowing capillary action to draw the infiltrant medium into the part above the liquid level, and then “blotting” the infiltrated part on an absorbent material to remove excess infiltrant from the macropores. However, this technique still has not provided as much control as might be desired over where and in what quantity an infiltrant material is placed within the porous preform.

White also briefly discloses a pipetting method, but does not teach using any particular relationship between the volume of pipetted material added as compared to the available void volume of the preform, and did not achieve the distribution that he sought of gelatin in the matrix. In this method, the volume of infiltrant added to the preform was not measured or controlled. In particular, White's technique resulted in essentially all surfaces of all pores being at least coated with infiltrant material, even in the case of pores that in the finished product were mostly free of infiltrant material. This was so because at a certain point during White's manufacturing process, all pores were substantially fully occupied by liquid infiltrant, and only at a later step was some of the liquid infiltrant removed from some of the pores by blotting. Having been once exposed to liquid infiltrant, the pores could not be made completely infiltrant-free after that. Such a biostructure has had a shortcoming in that surfaces which have even a thin coating of polymer may be less conducive to ingrowth of natural bone and tissue than a bare surface of an osteoconductive preform material would be.

Accordingly, it may be desirable to provide a biostructure having pores at least some of which are partially but not completely occupied by an interpenetrant. It would be desirable to have some of the pores not exposed to any of the interpenetrant, not even in the form of a coating on the walls of the pores. It would further be desirable that the pores which are unexposed to the interpenetrant could be located not just on the overall exterior surface of the biostructure, but also at least on interior surfaces which define the boundaries of possible macroscopic internal features in or through the biostructure.

It would be desirable to provide a biostructure which exhibits a gradient or variation from one region or portion of the biostructure to another, in terms of the extent to which pores are occupied by the interpenetrant, and in general, it would be desirable to be able to vary the extent of occupancy of pores by the interpenetrant from place to place within a biostructure. It would be desirable to provide a gradient or variation of the composition of the interpenetrant from place to place within a biostructure. It would be desirable to provide as much geometric complexity of the biostructure as desired, including channels there through. It would be desirable to provide appropriate methods of manufacturing any such biostructure.

### BRIEF SUMMARY OF THE INVENTION

The present invention is directed toward a biostructure comprising a matrix having pores; the pores of the matrix being either partly or fully occupied in some but not all places by an interpenetrant. On at least some surfaces of the biostructure, the biostructure may have pores that are substantially free of the interpenetrant. Similarly, the biostructure may have such unoccupied pores along surfaces that define macroscopic internal features within the matrix. The extent of filling by the interpenetrant may vary as a function of the size of the pores and may vary as a function of the region of the biostructure in which particular pores are located, and may vary as a function of whether or not particular pores are on a surface of the biostructure. The composition of the interpenetrant may also vary from place to place. The biostructure may have some external surfaces which are penetrated by macro-channels while having other external surface(s) not penetrated by macro-channels, and may further comprise a lip. This aspect of the invention may be used even without interpenetrant. The invention also comprises methods of manufacturing the biostructures.

The biostructure may be an implantable bone substitute including but not limited to a bone repair device, a cranioplasty device, a burr hole cover or cap, a mandibular repair device, other craniofacial repair device, an alveolar ridge augmentation, a bone void filler, a spinal fusion or other spinal repair device, or other substitute for either a portion of a bone or an entire bone. The biostructure, or its corresponding matrix, may have dimensions which may be customized for a particular patient and which may be based on medical imaging data and may further include geometric features not present in the medical imaging data. The biostructure may be used in culturing cells outside the body of a patient.

### BRIEF DESCRIPTION OF THE FIGURES

The patent or application file contains at least one drawing executed in color. Copies of this patent or patent application publication with color drawing(s) will be provided by the Office upon request and payment of the necessary fee.

FIG. 1 is a schematic illustration of a biostructure of the present invention in which pores at some of the external surface and pores at the boundary of a macroscopic internal feature are free of an interpenetrant in accordance with principles of the present invention.

FIG. 2 is a schematic illustration of a biostructure of the present invention having a pore size distribution, illustrating smaller interstitial pores being more fully occupied by the interpenetrant than larger pores in accordance with principles of the present invention.

FIG. 3 is a schematic illustration of a biostructure of the present invention having a gradient of pore occupancy by the interpenetrant, with the pores at a top end of the biostructure being more fully occupied than pores at a bottom end of the biostructure in accordance with principles of the present invention.

FIGS. 4A-C are schematic illustrations of methods of creating a biostructure having a gradient of pore occupancy by the interpenetrant in accordance with principles of the present invention.

FIG. 5 illustrates manual pipetting for dispensing liquid infiltrant into a matrix in accordance with principles of the present invention.

FIG. 6 illustrates a CAD model of a biostructure made in Example 1 in accordance with principles of the present invention.

FIG. 7A is a photograph of the exterior of an entire biostructure made in Example 1, and FIG. 7B is a Scanning Electron Microscope (SEM) micrograph of a portion of the exterior surface of the same biostructure in accordance with principles of the present invention.

FIGS. 8A, 8B and 8C show the CAD model or mathematical sections through the CAD model, which are used to illustrate where the physical sectioning was performed through the biostructures made in Example 1. FIGS. 8D and 8E are SEM micrographs which depict the sections diagrammed in FIGS. 8A through 8C in accordance with principles of the present invention.

FIG. 9 illustrates measured mechanical strength of the biostructures of Example 1, as a function of extent of occupancy of the pores by the interpenetrant in accordance with principles of the present invention.

FIG. 10A shows a photograph of a histology section of a burr hole cover that was implanted in an animal for four months. FIG. 10B shows a magnified version of that same image in accordance with principles of the present invention.

FIG. 11 shows a photograph of a histology section of a burr hole cover that was implanted in an animal for six months in accordance with principles of the present invention.

FIG. 12 shows, for comparison, a photograph of a histology section of a burr hole cover with only hydroxyapatite and no interpenetrant, four months post-implantation in accordance with principles of the present invention.

FIGS. 13A-C show a geometry of a burr hole cover which has a lip as an aid in positioning and fixating in accordance with principles of the present invention.

### DETAILED DESCRIPTION OF THE INVENTION

The invention includes both a biostructure and a method of manufacturing the biostructure. The biostructure may be implanted as a prosthesis or bone replacement device. The biostructure may be an implantable bone substitute including but not limited to a bone repair device, a cranioplasty device, a burr hole cover or cap, a mandibular repair device, other craniofacial repair device, an alveolar ridge augmentation, a bone void filler, a spinal fusion or other spinal repair device, or other substitute for either a portion of a bone or an entire bone. The biostructure, or its corresponding matrix, may have dimensions which may be customized for a particular patient and which may be based on medical imaging data and may further include geometric features not present in the medical imaging data. The biostructure may be used in culturing cells outside the body of a patient.

Article of Manufacture

The invention includes a biostructure having a matrix that may be a network such as a three-dimensionally interconnected network. The matrix may define interstitial pores and the pores may have a size or size distribution that is appropriate to encourage the ingrowth of bone or other tissue. For example, the pores may have most of the pore volume being contained in pores whose dimension is in the range of 1 to 100 micrometers. More particularly, the pores may have most of the pore volume being contained in pores whose dimension is in the range of 8 to 12 micrometers. The matrix may be made of particles that are partly joined to each other. To the extent that the particles are identifiable as nearly discrete particles, and excluding the necks which may join particles to other particles, the particles may have average overall dimensions which are somewhere between one and two times the pore dimension.

The matrix may be such that it has a matrix density (the weight of the matrix divided by the overall volume of the matrix, which include the volume of pores and the volume of solid matrix material), which is in the range of approximately 50% to approximately 80% of the full solid density (“true” density) of the material of which the matrix is made.

The matrix may also define macroscopic internal features such as passageways, channels, or other features having a size scale that is somewhat larger than the dimension of the pores. For example, these macroscopic internal features may have cross-sectional dimensions in a range such as from 100 micrometers to 1000 micrometers. More particularly, the macroscopic internal features may have cross-sectional dimensions in a range such as from 400 micrometers to 600 micrometers. The macroscopic internal features may be passageways, channels or other features, may be either through the biostructure or dead-ended, may include branchings or intersections with other macroscopic internal features, may have constant or variable cross-section, and may be straight or non-straight, in any combination of these attributes. Such macroscopic internal features may be chosen to be of appropriate size and geometry to encourage the ingrowth of blood vessels which can supply nutrients to and remove waste products from cells, or may be chosen so as to be appropriate to serve as a route for rapid advancement of ingrowing bone or tissue into the implant. The matrix also may have almost any degree of geometric complexity including overhangs and undercuts.

The pores may be at least partially occupied in at least some places by an interpenetrant that may be a material capable of being hardened from a liquid state or from a liquid substance. The overall volume of interpenetrant may be less than the total volume of pores in the matrix.

If macroscopic internal features such as passageways, channels, or other such features are present in the biostructure, the occupation of space by the interpenetrant may be such that the macroscopic internal features may be substantially free of the interpenetrant in their overall cross-sectional empty space.

Within portions of the biostructure that do contain matrix, as opposed to being macroscopic internal features, the occupation of space by the interpenetrant may be such that at least some regions of the biostructure is matrix having pores that are free of the interpenetrant. This region or regions that are free of the interpenetrant may be on the overall external surface of the biostructure. However, it is also possible that there may be at least one place at the overall external surface of the biostructure in which pores at the external surface are coated with the interpenetrant, as a consequence of manufacturing techniques described elsewhere herein. The biostructure may include pores that are only partially occupied by the interpenetrant.

If macroscopic internal features such as passageways, channels, or other such features are present in the biostructure, the occupation of space by the interpenetrant may be such that at least some surfaces of the matrix that bound or define the macroscopic internal features might neither contain nor be coated by the interpenetrant. Alternatively, even if such bounding surfaces do contain some of the interpenetrant, they might contain less of the interpenetrant than is found elsewhere inside the biostructure. As a result, the interior surfaces which form the boundaries of macroscopic internal features may benefit (have improved ability to promote bone and tissue ingrowth) because of having surface pores which are completely free of the interpenetrant or which contain less of the interpenetrant than regions elsewhere such as within the bulk of the biostructure. This would be similar to the reason why the overall external surface of the biostructure is known to benefit, have improved ability to promote bone and tissue ingrowth, as a result of such absence of interpenetrant. However, although it is believed to be desirable, it is not required that all of these interior surfaces at a macroscopic internal features level be free of the interpenetrant.

In the biostructure of the present invention, there may be regions that may be completely free of the interpenetrant. Such regions can include interior regions as well as regions at the overall external surface of the biostructure. The biostructure may have regions which, averaged over a suitable space, contain different amounts of the interpenetrant or whose pores are occupied to different extents by the interpenetrant, as compared to other regions of the same biostructure. The arrangement may be such as to exhibit a gradient, from one region of the biostructure to another, in terms of the extent of occupancy by the interpenetrant. These regions that are completely free of the interpenetrant or have differing amounts of interpenetrant may be distributed as desired within the biostructure, limited only by techniques and access points as described elsewhere herein.

In the biostructure of the present invention, the matrix may have a distribution of pore sizes ranging from smaller to larger size pores. In general, the smaller size pores may have a larger fraction of their empty volume occupied by the interpenetrant than do the larger pores. The fractional extent of occupancy of pore space by the interpenetrant may decrease with increasing pore size.

The matrix may be made of or may include substances that resemble or are compatible with natural bone. The matrix may be osteoconductive or even osteoinductive. The matrix may be made of or may include one or more ceramic substances, such as one or more members of the calcium phosphate family. The matrix may be either resorbable or nonresorbable by the body or, if made of more than one substance, may be made of both resorbable and nonresorbable substances.

Among calcium phosphates, hydroxyapatite is generally considered nonresorbable, while tricalcium phosphate is resorbable. Tricalcium phosphate may include either one or both of the known crystal structures (alpha and beta) of tricalcium phosphate, in any proportion. For example, the matrix may be made of a combination of hydroxyapatite and tricalcium phosphate. The matrix may be made of or may include calcium sulfate. The matrix may be made of or may include bioactive glass.

In the case of some materials, such as ceramics, the particles may be joined to each other by necks that are substantially the same material as the particles themselves. It is also possible that the particles might be joined to each other by necks that may comprise a binder substance different from the substance of which the particles themselves are made. For example, the material of which the matrix is made may be or may include particles of demineralized bone matrix. In that situation, the binding substance could be a substance such as collagen, gelatin, starch or related derivatives. If the matrix comprises more than one substance, those substances may be distributed in a defined geometric pattern or distribution. The matrix may also be made of other, non-osteoconductive material.

The interpenetrant may have mechanical properties that are suitable so that when the matrix and the interpenetrant form an interpenetrating phase composite or interlocking networks, the combination results in a mechanical property that is modified in a desirable way, such as by having increased strength or fracture toughness. The interpenetrant may be or may include one or more polymers. The polymer(s) may be either resorbable or nonresorbable or a combination thereof. Polymethylmethacrylate (PMMA) is an example of a nonresorbable polymer. Poly lactic acid (PLA) and poly lactic co-glycolic acid (PLGA) are examples of resorbable polymers. Polycaprolactone (PCL) is another example of a resorbable polymer.

The interpenetrant may include any of various types of activators, initiators, catalysts, etc., suitable to promote the transformation of monomer to polymer, if the manufacturing method involves a step of transforming monomer to polymer. The interpenetrant may be or may include a comb polymer.

The interpenetrant does not need to be of identical composition from place to place within the biostructure. The composition of the interpenetrant could vary from place to place within the biostructure. The resorbability or resorption rate of the interpenetrant could vary from place to place within the biostructure.

The biostructure may be an implantable bone substitute including but not limited to a bone repair device, a cranioplasty device, a burr hole cover or cap, a mandibular repair device, other craniofacial repair device, an alveolar ridge augmentation, a bone void filler, a spinal fusion or other spinal repair device, or other substitute for either a portion of a bone or an entire bone. The biostructure, or its corresponding matrix, may have dimensions which may be customized for a particular patient and that may be based on medical imaging data and may further include geometric features not present in the medical imaging data. The biostructure may be used in culturing cells outside the body of a patient.

It is also possible that space not occupied by either the matrix or the interpenetrant could be occupied, either partially or completely, by yet another material, which may be designated as a third material. The third material may belong to any category, including categories of materials other than the categories to which the matrix material and the interpenetrant belong. For example, the third material may be a dissolvable material such as a water-soluble material, which may, for example, be chosen to provide protection to the matrix during handling, during surgical installation, etc. The solubility of a dissolvable material in water, which is representative of bodily fluids, is one factor which influences how long the dissolvable material will remain in the implant after its implantation into the body of a patient. A dissolvable material may be chosen to have an appropriate solubility in water at physiological conditions. It is also possible that the third material may be chosen to be an Active Pharmaceutical Ingredient, an anesthetic, an antibiotic, an anti-inflammatory, a chemotherapeutic agent, growth factors, or other bioactive substance.

The biostructure may be sterile and may be appropriately packaged so as to remain sterile.

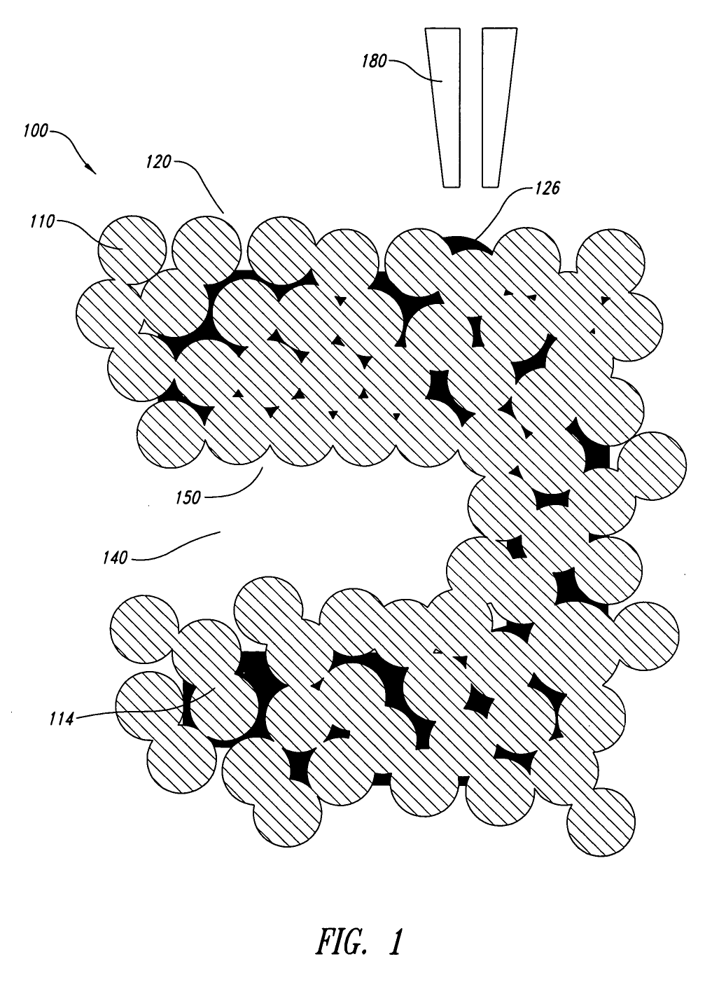

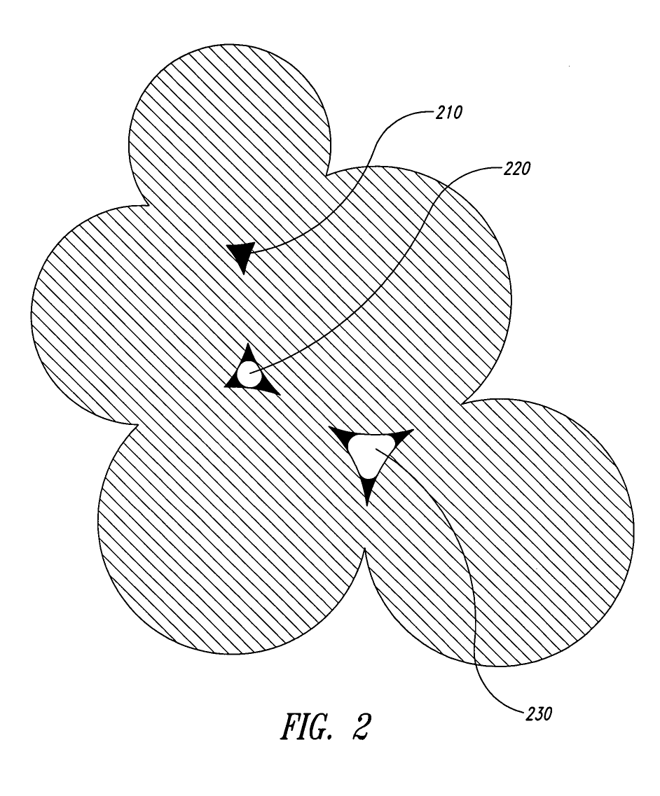

A biostructure of the present invention is further illustrated in FIG. 1, which is a cross-section of the biostructure. The biostructure 100 may comprise a plurality of particles 110 which may be partially joined to each other. In FIG. 1 the particles 110 are shown as being joined directly to each other, i.e., the necks 114 comprise substantially the same substance as the particles 110 themselves. Alternatively, it is possible that the particles may be joined to each other by necks that are a binding substance (not illustrated) that are different from the substance the particles themselves are made.

The biostructure 100 has an overall external surface 120. The external surface 120 is shown as, in some regions, having a porous surface that is not coated by the interpenetrant. A place 126 at the overall external surface of the biostructure in which pores at the external surface are coated with the interpenetrant is also shown in FIG. 1.

The biostructure 100 also is shown as containing or defining a macroscopic internal feature, such as macrochannel 140 in FIG. 1 that is shown as a dead-end macrochannel. In FIG. 1 the macrochannel 140 is shown as not being occupied by the interpenetrant. Furthermore, in FIG. 1 the pores 150 of the biostructure that bound and define the macrochannel 140 are also shown as not being occupied by or coated with the interpenetrant.

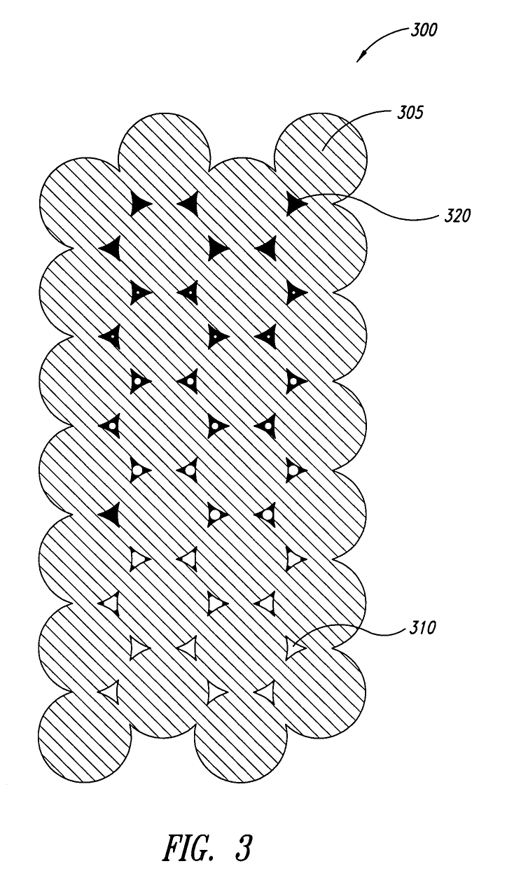

It is possible that the biostructure may have a distribution of pore sizes, as illustrated in FIG. 2. If the overall fraction of pore space that is occupied by the interpenetrant is not very close to unity, it is likely that many pores will be less than fully occupied by the interpenetrant. It is possible that pores of relatively smaller pore size may be more completely occupied by the interpenetrant, while pores of relatively larger pore size may be less completely occupied by the interpenetrant. The fractional extent of occupation of pore space by the interpenetrant may decrease with increasing pore size.

A matrix with a distribution of pore sizes is shown in FIG. 2, which, for clarity of illustration, shows only a small number of pores. Pores of three different sizes are shown in FIG. 2. The smallest pore 210 is shown as being completely occupied by interpenetrant. The medium sized pore 220 is shown as being somewhat occupied by interpenetrant, and the largest pore 230 is shown as having the smallest fraction of its volume occupied by the interpenetrant. In the biostructure of the present invention, the situation illustrated in FIG. 2 may be combined with the situation where pores or incomplete pores at a bounding surface remain not occupied by the interpenetrant. A bounding surface may refer either to the overall external surface 120 of the biostructure 100 or to the surface 150 that bounds a macroscopic internal feature 140 within the biostructure 100.

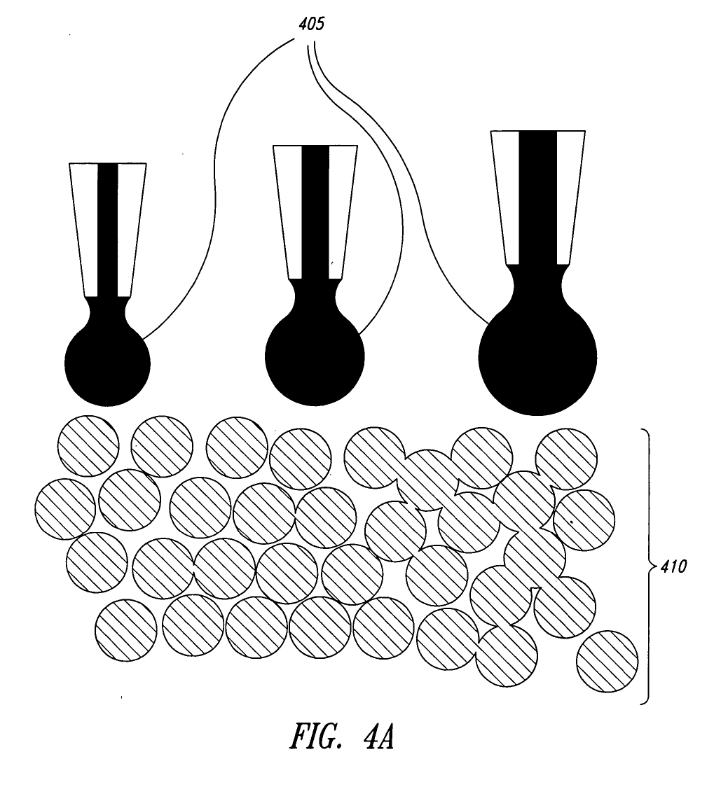

Another possible aspect of a biostructure of the present invention is illustrated in FIG. 3. For simplicity of illustration, in FIG. 3, all of the particles 305 and all of the pores 310 are shown as being of identical size and spacing. The extent of occupancy of pores by the interpenetrant 320 is shown as varying from one place in the biostructure to another. At the top of the biostructure 300 in FIG. 3, pores are shown as being substantially fully occupied by the interpenetrant, while at the bottom of the biostructure in FIG. 3, pores are shown as being substantially empty of the interpenetrant. In between, FIG. 3 shows a variation in the extent to which pores are occupied by the interpenetrant.

A specific aspect of the invention is that the biostructure may be a bone substitute whose pores comprise a chemotherapeutic agent. This may be useful in situations where a bone or a portion of a bone must be removed due to cancer. The removed bone can be replaced by a bone substitute that also contains and locally delivers a chemotherapeutic substance. Local or site-specific delivery of such a substance can reduce detrimental effects on the body as a whole, while delivering required quantities at the site where the substance is needed. Such a biostructure may comprise any of the features described elsewhere herein such as particular pore size, mechanical strength, presence of macrochannels, choice of matrix material, presence of polymers as interpenetrants together with the bioactive substance which in this case is a chemotherapeutic agent, etc. However, these features are not essential limitations. One possibility is that the chemotherapeutic agent may be located in spaces not occupied by either the matrix or the interpenetrant. Another possibility is that the chemotherapeutic agent may be commingled with the interpenetrant.

Another specific aspect of the invention is that the biostructure may comprise an anesthetic substance in the same way as the just-described chemotherapeutic substance.

Another aspect of the invention is that the article as described herein may be a component of a kit. The kit may, for example, comprise tooling appropriately sized to create a defect that is dimensionally matched to the article itself.

Method of the Invention

The invention also comprises a method of manufacturing the described biostructure.

According to aspects of one embodiment of the present invention as a first step, a preform may be manufactured. The term preform may be considered to refer to a manufactured article prior to addition of liquid infiltrant. The preform may be manufactured by any appropriate manufacturing technique, which may include three-dimensional printing. In three-dimensional printing, powder particles may be joined together by a binder substance that may be dispensed in the form of a liquid, such as an aqueous solution of the binder substance. For certain manufacturing sequences, the binder substance may be chosen so as to be capable of decomposing into gaseous decomposition products at a temperature less than a sintering temperature of the matrix material.

Techniques for manufacturing the preform also may include sintering suitable to cause individual powder particles to join to each other in a way that still leaves some porosity within the preform. The preform may be manufactured so as to have macroscopic internal features such as channels or passageways or other features at a size scale larger than the size scale of the inter-particle porosity. The preform may be manufactured so as to contain other complex geometric features such as overhangs, undercuts, etc. The preform may be manufactured having variation of composition, which may use techniques such as are described in co-pending commonly assigned U.S. patent application Ser. No. 10/122,129 “Method and apparatus for engineered resorbable biostructures such as hydroxyapatite substrates for bone healing applications,” which is hereby incorporated by reference.

A next step may be to determine the amount of void volume in the preform, or, in greater detail, the amount of void volume as a function of the size of pores or empty features. If an approximate knowledge of the void volume is sufficient, the preform may be manufactured using parameters which are already known to result in a desired fraction of porosity and/or pore size distribution, and it might not be necessary to take a measurement after manufacturing of the specific biostructure or batch of biostructures.

Alternatively, at the time of completion of the steps involved in manufacturing the preform, it is possible to measure such parameters either for the biostructure being manufactured or for a similar article similarly manufactured such as from the same batch.

A general and non-destructive way is to determine the mass of the biostructure and the overall volume of the biostructure and to compare the ratio of those two quantities to the theoretical solid density (“true density”) of the matrix material. This may be done as simply as by using a balance for mass measurement and calipers for dimensional measurement.

A more specific way of measuring both the overall volume and the pore size distribution is mercury intrusion porosimetry. It may be desired that, if the preform contains macroscopic internal features, the volume of the macroscopic internal features not be counted as void volume for purposes of being partially occupied by the interpenetrant. Measurements by mercury intrusion porosimetry may be suited to such a determination because mercury intrusion porosimetry does not recognize pores or voids or empty spaces larger than a certain minimum size anyway. It is possible that even if the biostructure contains macroscopic internal features, the porosity fraction or parameters may be measured using surrogate biostructures that do not contain macroscopic internal features, and such measurements obtained using the surrogate may be used in setting manufacturing parameters for the actual biostructures.

A next step may be to decide what fraction of the void volume of the biostructure is desired to be occupied by liquid infiltrant and thereby calculate a desired volume of liquid infiltrant to be dispensed into the preform. The chosen amount of liquid infiltrant may be chosen to be less than the total pore volume of the preform or may be chosen to be a desired fraction of the total pore volume of the preform.

Alternatively, instead of being based on the total pore volume of the preform, the chosen amount of liquid infiltrant may be chosen based on the total volume of pores whose size is less than a certain pore dimension. The total pore volume may be calculated excluding the void volume of macroscopic empty features that may be present in the preform. In order to achieve bounding surfaces that are substantially free of interpenetrant, it may be desirable that the chosen volume of liquid infiltrant be less than approximately 80% of the total volume of pores, not counting the volume of macroscopic internal features, in the biostructure.

A step that can optionally be performed before actual infiltration by liquid infiltrant is to treat the preform with a coupling agent that may be suitable to improve the eventual bond between the interpenetrant and the matrix, such as by chemically preparing pore surfaces to result in improved adhesion. Suitable coupling agents include silanes and titanates, as is known in the relevant art. It may be desirable to include the coupling agent in the formula for the interpenetrant, which would reduce the number of manufacturing steps.

A next step may be to dispense onto the preform in selected places a liquid infiltrant. The liquid infiltrant may contain the interpenetrant or may be capable of transforming into the interpenetrant, such as by chemical change. The liquid infiltrant may be capable of hardening into a solid interpenetrant after its infiltration into the preform. The liquid infiltrant may be a monomer, or may be a solution of polymer in monomer. A monomer or monomer-containing liquid infiltrant may further comprise any of various types of activators, initiators, catalysts, etc., suitable to promote the transformation of monomer into polymer.

Another possibility is that the liquid infiltrant may be a solution of polymer in a solvent that is capable of evaporating. The liquid infiltrant may be chosen to have a viscosity suitable for infiltrating into the described pores. A suitable viscosity range may be from approximately 1000 centipoise as a rough upper limit, down to as a lower limit, the viscosity of the lowest-viscosity liquid typically used as a solvent, which is slightly under 1 centipoise. This viscosity range is quite broad and encompasses many liquids. (1 Poise=1 dyne-s/cm²; 1 centipoise=1 milliPascal-second) The viscosity range may be even broader depending on preform design, e.g., biostructures with many macrochannels are more easily infused than those without. The liquid infiltrant may further contain any one or more of a water-soluble substance, an Active Pharmaceutical Ingredient, an anesthetic, an antibiotic, an anti-inflammatory, a chemotherapeutic agent, growth factors, or other bioactive substances, etc., in any combination.

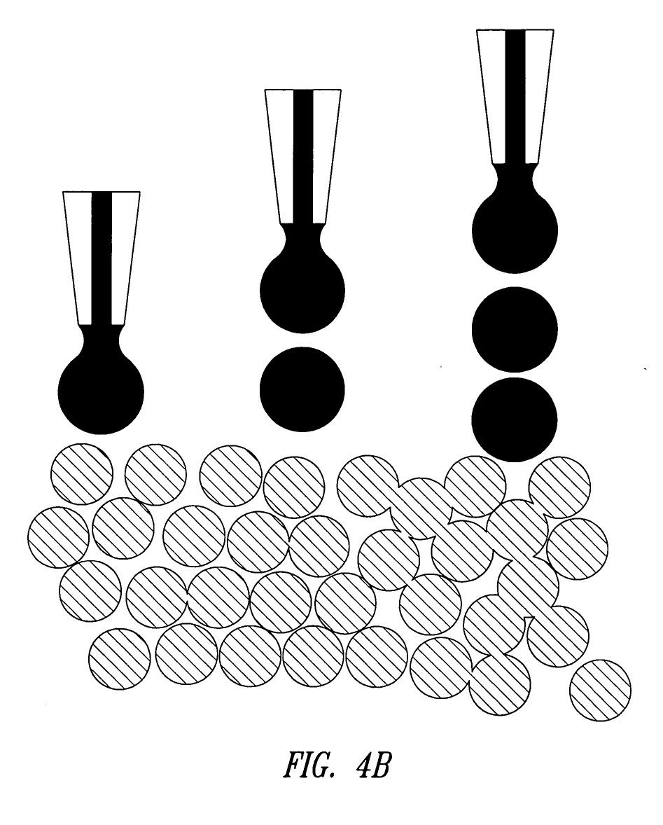

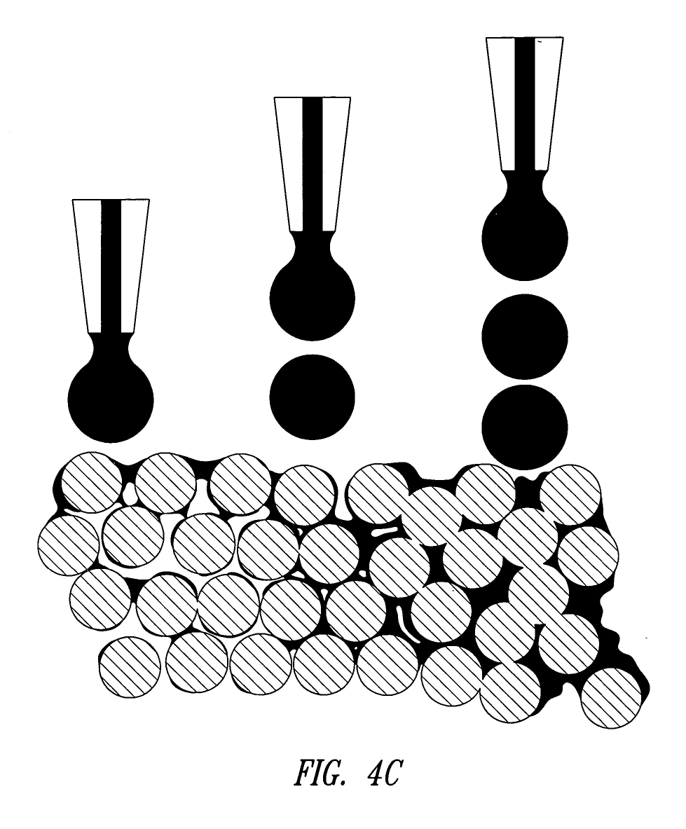

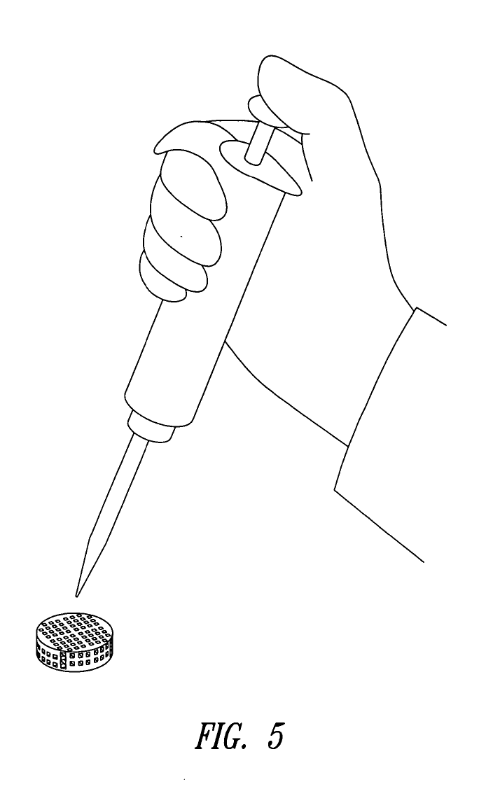

The liquid infiltrant may be applied onto selected places on the preform, by means of a dispensing device such as dispenser 180 shown schematically in FIGS. 1 and 4. In its simplest form, the dispensing device may be a hand-operated dispensing device such as a micropipette, which is shown in FIG. 5. Such micropipettes may be adjustable as to the amount of liquid that they take up and then dispense. Dispensing of relatively viscous liquids may include the use of a correction factor, which may be calibrated, reflecting the fact that some liquid may remain on surfaces of the micropipette. Other liquid metering apparatus may also be used, as those skilled in the art will appreciate. In other practices of the invention, the dispensing may be more automated in terms of either physical placement of the liquid infiltrant or amount of liquid infiltrant dispensed or both.

Dispensing of liquid infiltrant can be performed at more than one place on the biostructure being infused, with different amounts of liquid infiltrant being dispensed in individual places, as desired. Dispensing may comprise dispensing a predetermined total amount of liquid infiltrant into the biostructure, or dispensing predetermined individual amounts of liquid infiltrant into predetermined places of the biostructure. During dispensing, record may be kept of the amount of liquid infiltrant dispensed at any given location and of the total amount of liquid infiltrant dispensed for the entire biostructure. This information may be compared to predetermined intended amounts of liquid infiltrant. The ease of keeping some pores completely dry (free of liquid infiltrant) is influenced by the total amount of liquid infiltrant compared to the total pore volume. Smaller liquid infiltration fraction makes it easier to keep pores, or certain pores, dry (free of liquid infiltrant).

It is known that when a liquid infiltrates into a porous solid having a distribution of pore sizes, there is a tendency for the liquid to preferentially fill smaller pores before the liquid fills larger pores. This occurs because at a free surface (liquid-gas interface), pressure created due to surface tension is stronger for small dimension pores than for large dimension pores. Therefore, the liquid is attracted into smaller dimension pores more strongly than the liquid is attracted into larger dimension pores. It is therefore possible to make a biostructure in which smaller pores are more fully occupied than larger pores, by manufacturing a biostructure having a pore size distribution, and then infusing into it a liquid infiltrant in an amount less than sufficient to fill all of the pores.

A biostructure which has entire regions within the biostructure free of interpenetrant can be made by dispensing only a relatively small volume of liquid infiltrant (compared to the total pore volume), and the dispensing may possibly include dispensing that material some distance away from the particular region(s) which is desired to be free of liquid infiltrant (and eventually the interpenetrant).

Similarly, a biostructure having a gradient of the occupancy of its pores may be made by dispensing different amounts of liquid infiltrant onto different places of the biostructure, as is illustrated in FIG. 4A-C. This can be done by dispensing different sizes of drops of liquid infiltrant, or different numbers of substantially identical drops of liquid infiltrant, or by other techniques. It is believed, although it is not wished to be restricted to this explanation, that when liquid infiltrant 405 is dispensed onto a porous structure 410 at a single point, in an amount insufficient to completely saturate the entire porous structure, there results a gradient in the extent of occupying of pores by liquid infiltrant, such that the extent of occupancy decreases with distance away from the point of deposition, until at a sufficiently great distance away from the point of deposition there may be porous preform which receives zero liquid infiltrant. This may naturally create a gradient of extent of occupancy by the interpenetrant, with the extent of occupancy possibly being greater closer to the point of dispensing of the infiltrant liquid.

However, it is believed that due to migration and capillary action, even the point(s) on the surface of the biostructure at which liquid infiltrant was dispensed may end up less than completely occupied by liquid infiltrant, although they may retain at least a coating of liquid infiltrant. This depends on, among other factors the overall extent of occupancy by the liquid infiltrant in the biostructure.

Achieving a distribution of occupancy fraction can be done with dispensing at just one dispensing point, or it can be done with multiple dispensing points at selected locations, with each dispensing location receiving either the same or different amounts of liquid infiltrant, as desired to achieve a desired distribution of the extent of occupancy by the interpenetrant. Multiple dispensing locations can be uniformly or non-uniformly distributed in space. For example, dispensing a relatively large amount of liquid infiltrant at a single location may achieve a greater depth of infusion than dispensing the same total amount of liquid infiltrant at a number of more distributed locations (see FIGS. 4B and 4C). With dispensing at many individual locations, the distribution of interpenetrant in the finished biostructure may resemble the distribution of liquid infiltrant. Dispensing at multiple locations can also involve dispensing different substances at different locations, as described elsewhere herein.

It is also possible that vacuum can be used during the infusion process. One possibility is that the entire infusion can be carried out in an environment of reduced absolute gas pressure, which may serve to reduce the amount of gas potentially available to be trapped as gas bubbles in undesired places during infusion, and, when the biostructure is returned to ordinary atmospheric conditions, correspondingly reduce the volume of such bubbles which may be trapped. Another possibility is that vacuum may be applied locally as suction to influence the motion of the liquid infiltrant into and within the preform.

Foreknowledge of the typical total void volume for a set of parts allows for the deliberate selection of volumes of liquid infiltrant that may be substantially less than the total amount that could be contained in the matrix. Parts infiltrated in such a manner may be said to be “infiltrant-deficient”. This can result in the liquid infiltrant pulling itself into the bulk microporosity of the matrix, leaving the overall external surface and the surfaces bounding macroscopic internal features substantially free of liquid infiltrant, due to the deficiency of volume of liquid infiltrant relative to total void volume of the pores.

It is not necessary that the same composition of liquid infiltrant be used at every location where liquid infiltrant is applied to the preform. It is possible to apply different compositions of liquid infiltrant at different locations and thereby achieve a variation of composition of interpenetrant from one place to another within the biostructure. The liquid infiltrants may be selected, for, example, so as to produce a gradient in the biostructure as far as resorbability or resorption rate of the interpenetrant.

After the preform has been infused with liquid infiltrant, the preform containing liquid infiltrant may then be subjected to a heating step suitable to promote transformation of monomer to polymer, although such a step is not essential. It is possible that after infiltration, in preparation for heating, the preform may be enclosed in a bag suitable to contain vapors, and the bag may be sealed. Such use of a bag can help to retain the known amount of liquid infiltrant in the biostructure, thereby counteracting a possible tendency for some of the liquid infiltrant to evaporate, which could create uncertainty or variability in the actual amount of interpenetrant remaining in the biostructure. The preform could just as well be partially enclosed in an unsealed bag at some earlier stage, with the bag similarly being sealed before heating.

If desired, the biostructure may also be infused to any desired extent with yet another material, which may belong to any of still other categories of materials, as described elsewhere herein. For example, this other material may be a dissolvable material, an Active Pharmaceutical Ingredient, an anesthetic, an antibiotic, an anti-inflammatory, a chemotherapeutic substance, a growth factor, or other bioactive substance. This could be done as a last step, after the placement of the liquid infiltrant, or, alternatively, it could be done earlier.

It is not necessary that such introduction of another substance be done after the described introduction of the liquid infiltrant. It is also possible that any of the described substances could be mixed together with the liquid infiltrant, or co-dissolved with the interpenetrant in a common solvent, or introduced before the introduction of the liquid infiltrant, or introduced into regions of the preform other than where the liquid infiltrant is introduced.

It is also possible that a preform could be made, and a fugitive material could be infused into a specified region or regions of the preform, and then an infiltrant liquid could be introduced using the metered infusion method of the present invention into at least some regions not occupied by the fugitive material, and the infiltrant liquid could be allowed to harden or caused to harden, and then the fugitive material could be removed.

The present invention is further illustrated by the following nonlimiting example:

### EXAMPLE 1

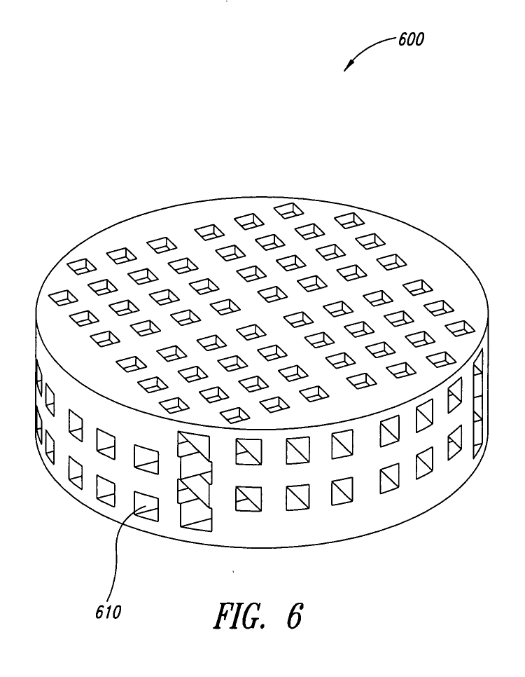

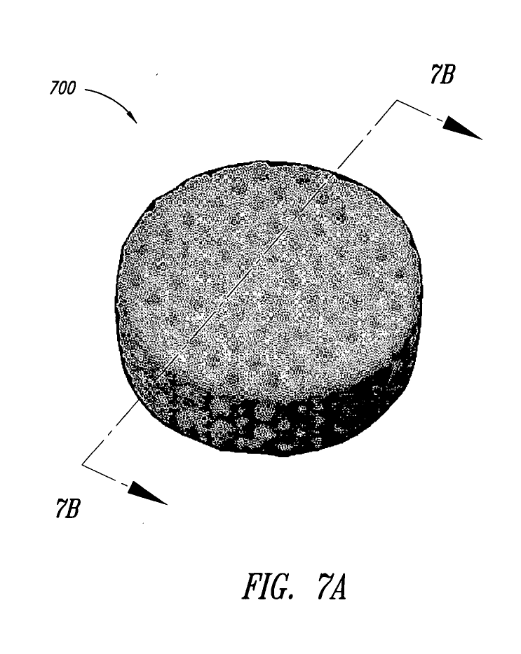

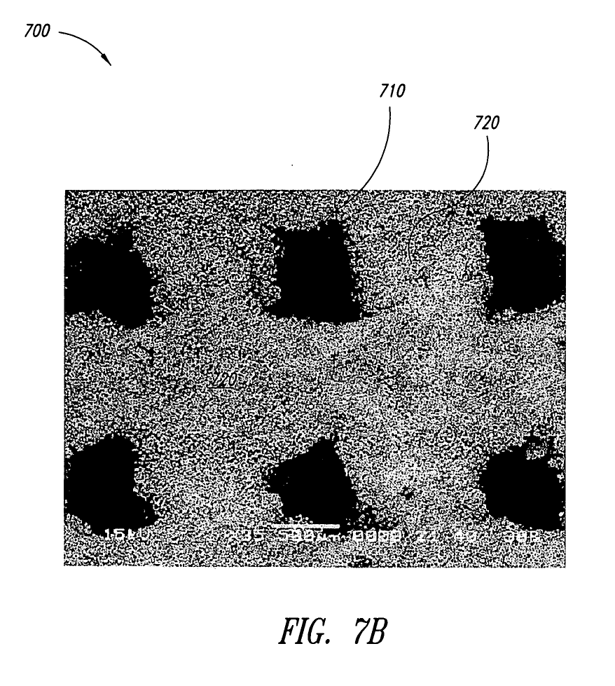

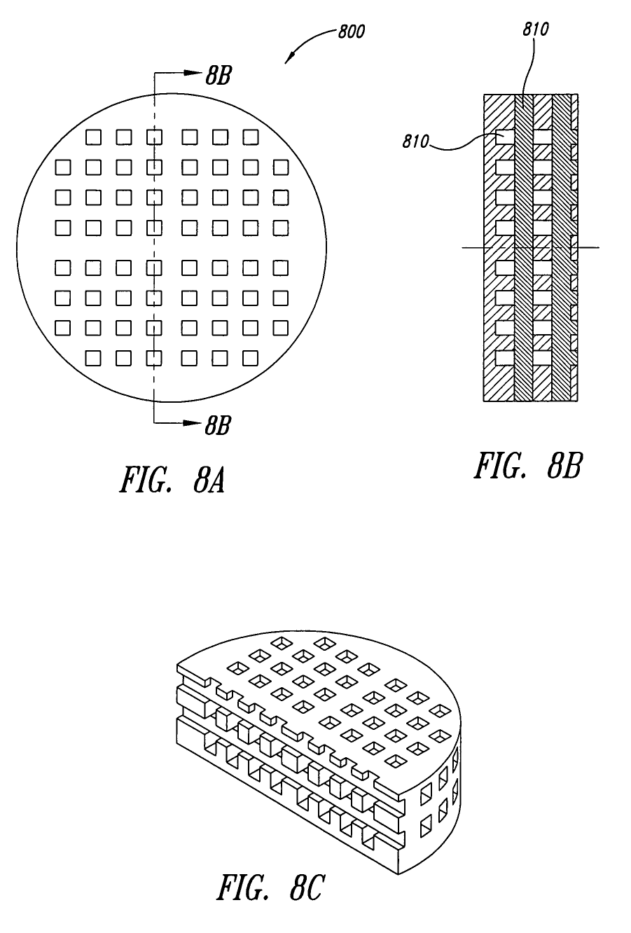

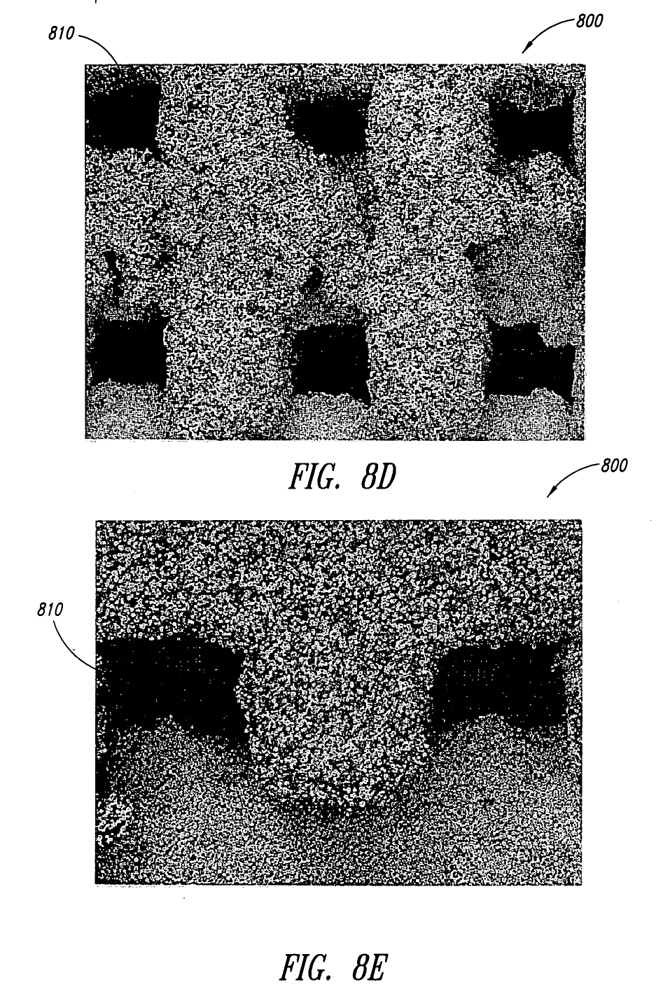

Porous biostructures were fabricated by three-dimensional printing starting from powder that was hydroxyapatite, followed by sintering. The powder particle size was about 25 micrometers. The binder substance used in the three dimensional printing was an aqueous solution of polyacrylic acid. Polyacrylic acid decomposes into gaseous decomposition products at a decomposition temperature lower than the sintering temperature of hydroxyapatite. The biostructure made in this Example also contained additional macroscopic internal features in the form of channels, which had cross-sectional dimensions of approximately 500 micrometers in each direction. The channels were present in the preforms as a result of the manufacturing of the preform by the three-dimensional printing process. The data reported here are from discs 600 of about 16 mm in diameter, 5 mm in height, with three sets of interior, interconnected, orthogonal macrochannels 610 of approximately 500 microns in cross-sectional dimension, as shown in the CAD solid model FIG. 6.

Similar hydroxyapatite biostructures have been characterized by mercury intrusion porosimetry, and were found to have continuous, interconnected micropores with most of the pore volume being in the pore size range of 8 to 12 micrometers. The void volume within the part, based on the microporosity alone, was about 44%. This refers to the porous solid portions of the part, i.e., not counting the interior space of the macrochannels.

The described discs had an overall geometric volume of 1071 mm³. The internal empty space of macrochannels was 377 mm³. The space occupied by the matrix, which was porous ceramic, was 673 mm³. Of the 673 mm³, 44% was pore space that on a small scale was empty (prior to infusion with polymer). The remaining 56% of the matrix was actual ceramic material. These fractions as measured by mercury intrusion porosimetry essentially treat the macrochannels as not being part of the biostructure, i.e., the empty space in the macrochannels is not counted as void volume and of course is not counted as solid volume either. Mercury intrusion porosimetry measures the void fraction and solid fraction of only the microporous regions. Mercury intrusion porosimetry cannot recognize or measure pores that are as large as the cross-sectional dimension of the macrochannels in this Example, because only minimal pressure is required to cause mercury to infiltrate features having the dimension of the macrochannels.

The method of the present invention was then used for the infiltration of porous hydroxyapatite (HA) discs. These parts were infiltrated in a measured, controlled manner using a micropipette as shown in FIG. 5 (available from Eppendorf, Hamburg, Germany; Brinkmann Instruments, Inc., Westbury, N.Y.). The infiltration was performed using a solution of 20% polymethylmethacrylate (PMMA) and 1% benzoyl peroxide in 79% methyl methacrylate monomer. These percentages are by weight. For the biostructures that are shown in SEM micrographs herein, the infusion fraction (the volume of liquid infiltrant dispensed into the biostructure, divided by the total volume of pores in the biostructure excluding the volume of the macrochannels) was between 40% and 60%.

After infusion, the parts were vacuum-sealed in bags made of polyvinyl alcohol and then were heated (under pressure) to complete polymerization of the monomer in the liquid infiltrant. The vacuum-sealing step was used to reduce evaporation and loss of methyl methacrylate, which is a volatile monomer.

FIG. 7A is a photograph of the entire exterior of the biostructure 700 made in this Example, and FIG. 7B is an SEM micrograph of a portion of the exterior surface. In FIG. 7B, the hydroxyapatite appears as lightly colored approximately spherical shapes, which is similar to the morphology of the powder used at the start of the three dimensional printing process. Dimensionally, the powder particles are approximately 25 microns in size. Some small “necks” are also visible connecting particles, which is a result of the sintering process. Also visible in FIG. 7B are several large, approximately square dark regions 710 that are the designed macrochannels, about 500 microns in cross-sectional dimension, which are empty space.

The salient feature of FIG. 7B is that the surface features 720 are mostly white, which indicates the presence of exposed hydroxyapatite powder particles that have not been coated by or exposed to interpenetrant. In FIG. 7B the existence of a predominantly hydroxyapatite exterior surface is indicated by the presence of many light-colored, interconnected spheres with little or no darker infilling between the spheres. In fact, a photograph of totally uninfused sintered hydroxyapatite, containing no interpenetrant at all, would look very similar to FIG. 7B.

In order to further display the characteristics of the manufactured parts, and to indicate further details of the achieved infiltration, selected biostructures 800 were physically sectioned along chords of the circular faces in an orientation as illustrated in FIGS. 8A, 8B and 8C. FIGS. 8A, 8B, and 8C use the CAD solid model to show with mathematical sectioning where the physical sectioning was performed on the actual biostructure. The sectioning was approximately parallel to two sets of channels 810, and perpendicular to the third set, and thereby reveals the interior channels along the cut.

FIGS. 8D and 8E are SEM micrographs of the biostructure after it had been sectioned as illustrated in FIGS. 8A-8C. FIGS. 8D and 8E are taken of slightly different places of the same sectioned biostructure 800, with FIG. 8E being a somewhat closer-in view than FIG. 8D. In both FIG. 8D and FIG. 8E, the large, square-like dark regions 810 are the designed macrochannels, about 500 microns in cross-sectional dimension, which are empty space. In order to appreciate what is shown by FIGS. 8D and 8E, it is necessary to contrast surfaces which are cut sections and surfaces which are the as-manufactured boundaries of macrochannels.

FIGS. 8D and 8E each show some surfaces that are cut sections and some surfaces which are the as-manufactured boundaries of macrochannels. It can be seen that physically cut sections have a degree of grayness, i.e., spherical powder particles are white as in FIG. 7B, but the spaces between spherical powder particles are distinctly dark. The darkness represents the interpenetrant, which in this case is PMMA. In the same photograph, however there is readily available a contrast.

According to the teachings of the present invention, the as-manufactured boundaries or interior surfaces of the macro-channels should be relatively devoid of dark interpenetrant material and should resemble the external surfaces as shown in FIG. 7B. It can be seen that in fact, those surfaces, which bound macrochannels, indeed are quite lightly colored even between individual powder particles, and therefore do resemble the overall exterior surfaces shown in FIG. 7B. This indicates that the infusion process of the present invention has kept those macrochannel-bounding surfaces substantially free of interpenetrant.

A study was performed concerning the mechanical effect of varying amounts of infiltrant in the preform. Preforms of identical design were infiltrated with various different quantities of infiltrant, so as to provide a range of fractional infiltration from only slight filling of pores at the low end of the range, to nearly complete filling of pores at the high end of the range. Data were collected as to the weight of these parts before and after infiltration. These data were used to estimate the fraction of the micropore void space infused with PMMA for each specimen, which is the calculated volume of infusing material divided by the volume of void space in pores having a size range of approximately 8 micrometers to approximately 12 micrometers.

The volume of interpenetrant material is determined from the change in weight of the biostructure, along with the known density of the interpenetrant material. The volume of void space in pores is known from porosimetry data. It can be noted that the actual amount of liquid infiltrant dispensed into the preform may differ from the nominal setting on the pipette due to viscous effects, and the difference may be characterized by a calibration curve. Similarly, the amount of infiltrant may differ due to evaporation of monomer during curing, which may be compensated for with a correction determined from experience.

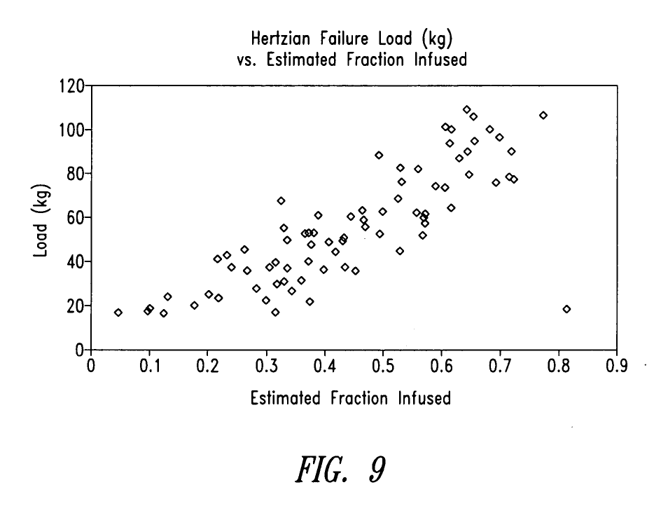

In preparing the plot in FIG. 9, the horizontal axis was calculated to represent what fraction of the void space was occupied by infiltrant material. The abscissas of plotted data points were calculated as

$$\begin{matrix}InfFrac = \frac{{Vol}_{PMMA}}{{Vol}_{Void}} \\ {Vol}_{PMMA} = \frac{{Mass}_{Infused} - {Mass}_{Initial}}{{ρ}_{PMMA}} \\ {Vol}_{Void} = \frac{{Mass}_{Initial} \cdot {Frac}_{Void}}{{ρ}_{HA} \cdot {Frac}_{Dense}}\end{matrix}$$

The true (solid) densities of HA and PMMA are known to be 3.14 and 1.19 g/cm³, respectively. The solid fraction and void fraction of the microporosity (0.56 and 0.44, respectively) were measured by mercury intrusion porosimetry.

The specimens were then subjected to load testing by Hertzian contact using a 0.25 inch (approx. 6 mm) diameter stainless steel ball probe, with the ball loading device pressing against the solid surface of the biostructure (i.e., the macrochannels were facing away from the surface which was loaded). Note that in FIG. 6, the bottom surface of the device in FIG. 6 is the surface that contains no macrochannels.

The peak loads at failure were recorded. In FIG. 9 these loads are shown plotted against the estimated fraction infused. There is some scatter, which may result from uncertainty in the determination of the infusion fraction, or from variation in the exact point of loading of individual discs, or from nonrepeatability of fracture data, or from any combination of these or other factors. Nevertheless, there is a clear trend of the data. The data in FIG. 9 illustrates that there is a monotonic increase in strength as a function of the infiltration fraction.

### EXAMPLE 2

Articles as made in the previous example were implanted in the crania of adult female sheep. Defects were made using a 16 mm diameter trephine. The burr-hole covers were 16.4 mm outside diameter and contained macrochannels whose cross-sectional dimensions were both approximately 500 micrometers. These particular burr-hole covers had no lips and were fixated by bone plates and screws. The burr-hole covers were made of hydroxyapatite and were infused such that approximately 40% to 60% of the total pore volume (not counting the volume of the macrochannels) was occupied by PMMA. The PMMA was polymerized in situ.

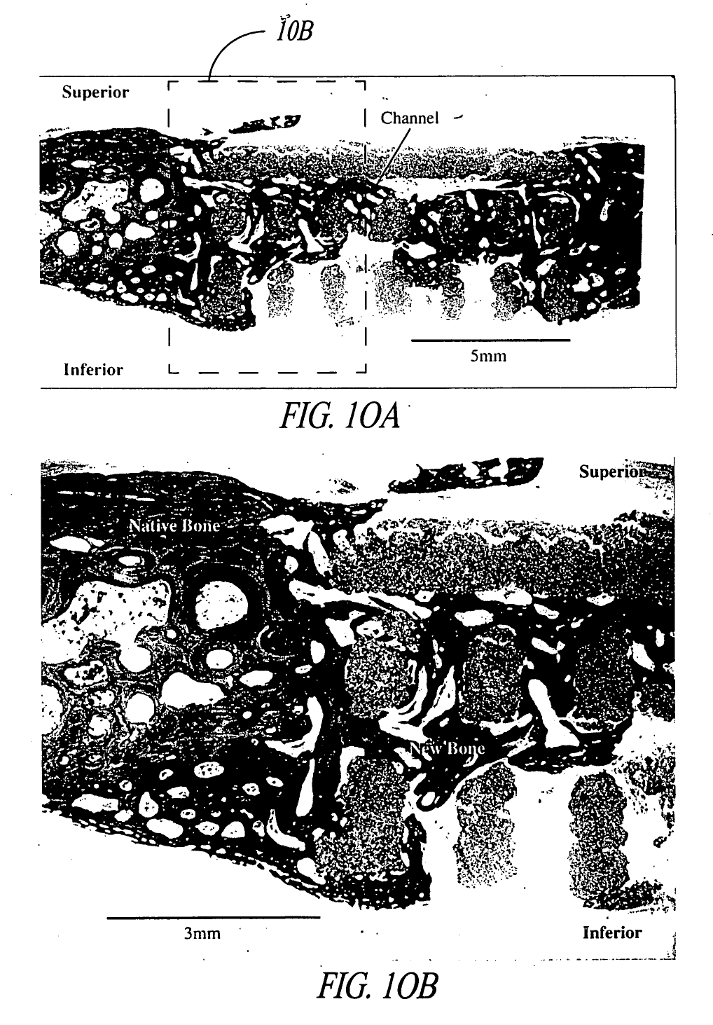

After implantation into the sheep, the animals were sacrificed at time points of 4 months and 6 months post-surgery, and histology results were obtained. Staining was performed using toluidine blue stain, which stains bone tissue blue. The HA/PMMA structure is visible in these photographs as gray. By looking at the gray regions, it is possible to see a cross-sectional structure of the burr hole cover similar to the CAD-generated cross-sections of FIGS. 8B and 8C. (In this Figure, the layer of the burr hole cover which is uninterrupted by macrochannels, which is the most external portion of the installed burr hole cover, is at the top of the illustration, which is different from its orientation in FIGS. 8B and 8C.) The mostly blue regions to the left and right of the structure of the burr hole cover are native bone. Histology results are shown in FIGS. 10A and 10B for the 4 month time point. In FIG. 10A, new bone formation can be observed extending through the channels within the device and bridging the defect.

In FIG. 10A the blue stained bone tissues almost bridge through the channels across the defect in the HA/PMMA device. Bone spicules are seen scattered at the dorsal ridge of the defect between the columns of fabricated channels. In FIG. 10A, the dashed rectangle indicates a smaller region which is shown in greater detail in FIG. 10B.

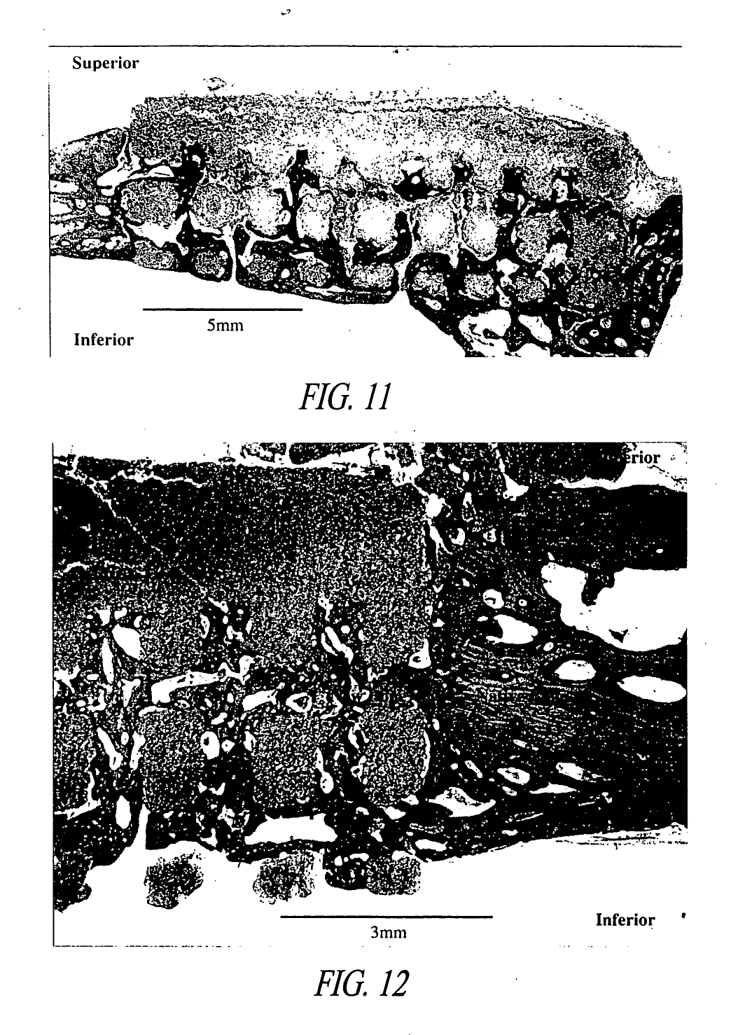

Next, FIG. 11 is a similar illustration of histology at the 6-month time point. In this photograph, extensive new bone formation can be observed extending throughout the channels of the device. The blue stained bone tissues almost bridge through the channels across the defect in the HA/PMMA device, where HA/PMMA appears as a globular mass. The connecting bone spicules stem from the defect margins into the channels. Bone spicules are seen scattered at the right dorsal ridge of the defect.

These results show significant, sustained bone growth both along the external surfaces and within the macrochannels of the devices. Both the extent of bone ingrowth and the close proximity of the new bone to the device surfaces suggest a substantial benefit from the presence of exposed, porous HA (not coated by polymer) on the exterior and channel surfaces of the burr hole cover of the present invention. The bone growth in the article of the present invention, as shown in these photographs, is comparable to what the ingrowth would have been into hydroxyapatite completely absent of PMMA.

For information and comparison, FIG. 12 shows histology of a completely uninfused hydroxyapatite burr hole cover, with no PMMA, at a four-month time point.

In general, the article of the present invention has the extra mechanical strength associated with the presence of the PMMA polymer. The article of the present invention is mechanically stronger than an uninfused ceramic article and yet retains most of the osteoconductive behavior of an uninfused ceramic article.

### EXAMPLE 3

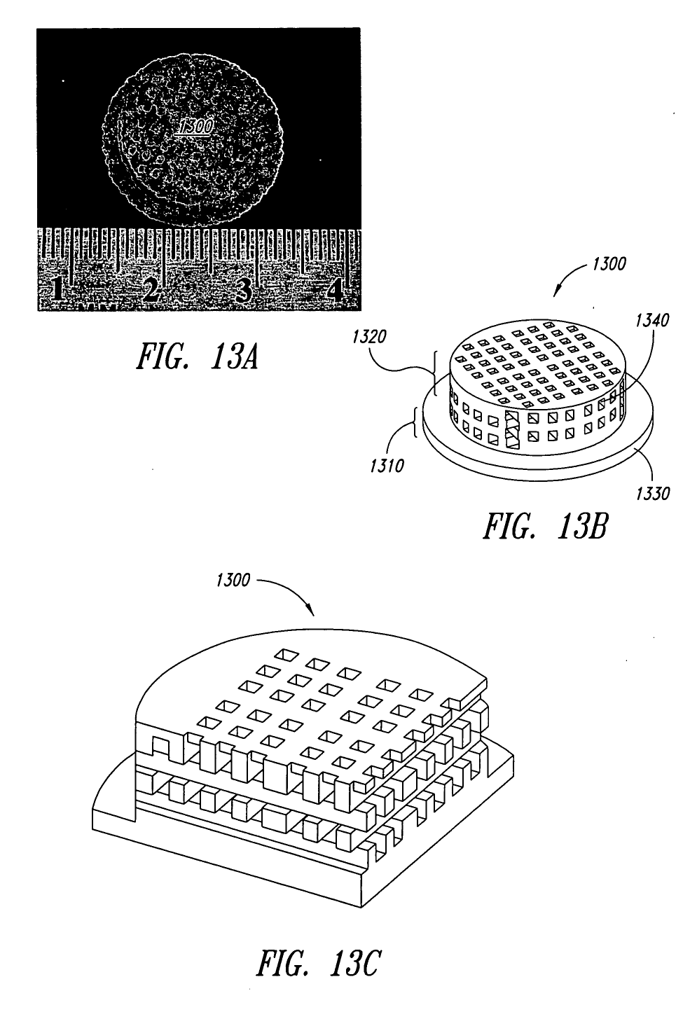

The invention includes the overall shape illustrated in FIGS. 13A-C, which may be a burr hole cover 1300 such as for cranial surgery. The article may have a first region 1310, which may be prismatic, connected to a second region 1320, which may also be prismatic, with the first region 1310 being everywhere larger than the second region 1320.

The first region 1310 may thus form a lip 1330 extending beyond the second region, with the lip 1330 being suitable for preventing the article from falling completely into or through a bone defect into which it is being placed. In the illustration, both regions are shown as being round, although other shapes are of course possible.

The second region 1320 may contain macro-channels 1340, which may be considered to be channels having at least one cross-sectional dimension in the range of 100 micrometers to 1000 micrometers. The first region 1310 may be free of macro-channels 1340. The first region 1310 may be intended to be the more exterior region upon placement in the body of the recipient. The first region 1310 may have an outward-facing surface which is either flat or curved (not shown), such as a curvature intended to correspond to the local curvature of the place in the recipient's body where it will be placed.

Both the first region 1310 and the second region 1320 may contain porosity. For example, both regions may be made of particles partly joined to each other. The macrochannels may define respective principal directions. Macrochannels may intersect other macrochannels in the form of an intersection between two macrochannels or even an intersection among three macrochannels. The macrochannels may be oriented such that the principal directions of macrochannels at an intersection point are substantially perpendicular to each other. Some macrochannels may begin and end at the exterior surface of the article, while others may have one end at the exterior surface of the article while having the other end inside the article, either dead-ended or at an intersection with another macrochannel.

The article may have one surface, which may in the installed condition be the surface facing the exterior of the patient, which may be substantially free of macrochannels. There may be porosity at this surface. This surface may be flat, or it may be curved, such as having a curvature similar to the local curvature of the part of the body where it will be implanted. There may be a penetration through this surface having dimensions and geometry suitable to permit the passage of a catheter from the external-facing side of the article through to the internal-facing side of the article.

Instead of having a lip as just described, it is also possible for the article to have other shapes which are larger at one end than at the other end so as to prevent the article from falling completely into or through a bone defect into which it is being placed. For example, the article may be a frustum of a cone, or may be a frustum of a pyramid.

The article as just described may contain interpenetrant as described elsewhere herein, or it does not have to contain interpenetrant.

Further Considerations and Summary and Advantages

The articles of the present invention can be used for filling a craniotomy or in general for filling any other bone defect of suitable size and shape, created for any reason.

It can be noted that in the method of the present invention, a fugitive material is not required. In the Example, no fugitive material was used for the creation/maintenance of a porous region during infiltration of an infiltrated part. This simplifies the manufacturing process.

The present invention is distinguished from that of White et al. in that the macroscopic internal features of the final device can be free of coating of infiltrant, which means that some micropores can be free of infiltrant, and individual regions can be free of infiltrant. The uninfused region is not limited to being on the generally exterior surface of the article, as in the case in Giordano. The present invention is also distinguished from both White and Giordano in that the macrochannels can be designed, having a desired detailed geometry and placement. It is also distinguished by the possibility of variation or gradients of interpenetrant occupancy fraction and of composition of interpenetrant. No blotting step is required, either.

For a given geometry and a given combination of materials (the matrix material used in making the preform and the interpenetrant used as the infiltrant) there is a range of final porosities (after infiltration) and associated mechanical strengths that may be achieved. A larger fraction of infiltration basically makes the biostructure stronger. This degree of freedom may prove useful in the tailoring of device properties to meet a desired biological function, which often requires a balancing of considerations concerning strength and porosity.

It can be noted that in the biostructure of the present invention, the placement of the interpenetrant can have a significant degree of localization. In particular, the bounding surfaces of internal channels, passageways and similar features can be kept free of interpenetrant if desired. The photographs in FIG. 8 indicate that the infusion process of the present invention has kept those macrochannel-bounding surfaces substantially free of interpenetrant. If one were to attempt to achieve a similar result by the method taught in Giordano, it would be extremely difficult to access the macrochannel-bounding surfaces for application of fugitive material. The achievement of interpenetrant-free macrochannel-bounding surfaces may be advantageous for applications in bone healing, so that bare (uncoated) matrix may be provided on the surfaces of the macroscopic internal features within the device, leading to better bone ingrowth and integration even at the localized size scale at which bone tissue penetrates from the macrochannels passageways and similar features into adjacent porous material of the implant. This is especially applicable in the case of an osteoconductive (or osteoinductive) matrix material. Previously, the ability to produce interpenetrant-free localized regions had been limited to mainly external surfaces of articles.

It can also be noted that in a biostructure in which completely infiltrated matrix is immediately next to completely uninfiltrated matrix (such as Giordano's article), there may be an additional stress concentration at that point of interface. In contrast, the biostructure of the present invention can provide a more gradual transition of mechanical properties that should result in less of a stress concentration.

All patents and patent applications and publications cited herein are incorporated by reference in their entirety. The above description of illustrated embodiments of the invention is not intended to be exhaustive or to limit the invention to the precise form disclosed. While specific embodiments of, and examples for, the invention are described herein for illustrative purposes, various equivalent modifications are possible within the scope of the invention, as those skilled in the relevant art will recognize. Aspects of the invention can be modified, if necessary, to employ the process, apparatuses and concepts of the various patents and applications described above to provide yet further embodiments of the invention. These and other changes can be made to the invention in light of the above detailed description. In general, in the following claims, the terms used should not be construed to limit the invention to the specific embodiments disclosed in the specification and the claims, but should be construed to include all biostructures that operate under the claims. Accordingly, the invention is not limited by the disclosure, but instead the scope of the invention is to be determined entirely by the following claims.

From the foregoing it will be appreciated that, although specific embodiments of the invention have been described herein for purposes of illustration, various modifications may be made without deviating from the spirit and scope of the invention. Accordingly, the invention is not limited except as by the appended claims

1. A biostructure that comprises a matrix that defines pores, wherein at least some pores are partially occupied by an interpenetrant, and wherein some pores are free of the interpenetrant.

2. The biostructure of claim 1, wherein at least some of the pores that are free of the interpenetrant are on an overall external surface of the biostructure.

3. The biostructure of claim 1, wherein at least some of the pores which are free of the interpenetrant are on surfaces which define macroscopic internal features of the biostructure.

4. The biostructure of claim 1, wherein the pores have a total pore volume, and the interpenetrant has a total interpenetrant volume, and the total interpenetrant volume is less than approximately 80% of the total pore volume.

5. The biostructure of claim 1, wherein the pores have a pore size distribution comprising various sizes of pores, and wherein smaller pores are occupied by the interpenetrant more completely than larger pores.

6. The biostructure of claim 1, wherein, within a region which is partially occupied by the interpenetrant, the fraction of occupancy of pore space by the interpenetrant varies from place to place.

7. The biostructure of claim 1, wherein the matrix comprises a ceramic.

8. The biostructure of claim 7, wherein the matrix comprises one or more members of the calcium phosphate family.

9. The biostructure of claim 1, wherein the matrix comprises hydroxyapatite.

10. The biostructure of claim 1, wherein the matrix comprises tricalcium phosphate.

11. The biostructure of claim 1, wherein the matrix comprises calcium sulfate.

12. The biostructure of claim 1, wherein the matrix comprises bioactive glass.

13. The biostructure of claim 1, wherein the matrix comprises demineralized bone matrix.

14. The biostructure of claim 1, wherein the matrix comprises more than one material distributed in a predetermined pattern.

15. The biostructure of claim 1, wherein the interpenetrant comprises at least one resorbable polymer.

16. The biostructure of claim 1, wherein the interpenetrant comprises at least one non-resorbable polymer.

17. The biostructure of claim 1, wherein the interpenetrant comprises at least one comb polymer.

18. The biostructure of claim 1, wherein the interpenetrant comprises polycaprolactone.

19. The biostructure of claim 1, wherein the interpenetrant is capable of hardening from a liquid state.

20. The biostructure of claim 1, wherein the interpenetrant comprises at least one activator or initiator or catalyst.

21. The biostructure of claim 1, wherein the interpenetrant has a composition that varies from place to place within the biostructure.

22. The biostructure of claim 21, wherein the compositional variation of the interpenetrant includes a variation in the resorption rate or resorbability of the interpenetrant.

23. The biostructure of claim 1, wherein the biostructure further comprises, in at least some space not occupied by either the matrix or the interpenetrant, a third material.

24. The biostructure of claim 23, wherein the third material is selected from the group consisting of water-soluble substances, Active Pharmaceutical Ingredients, antibiotics, anti-inflammatories, growth factors, other bioactive substances, chemotherapeutic agents, and anesthetics.

25. The biostructure of claim 1, wherein the matrix comprises particles partially joined to other particles.

26. The biostructure of claim 25, wherein the particles are joined to other particles by necks having a composition that is substantially the same as the composition of the particles.

27. The biostructure of claim 25, wherein the particles are joined to other particles by necks having a composition that is different from the composition of the particles.

28. The biostructure of claim 1, wherein the matrix has a pore size distribution of approximately 1 micrometer to approximately 100 micrometers.

29. The biostructure of claim 1, wherein a majority of pore volume is contained in pores having a pore dimension between 8 and 12 micrometers.

30. The biostructure of claim 1, wherein the matrix has a density of approximately 50% to approximately 80% of the solid density of the material of which the matrix is made.

31. The biostructure of claim 1, wherein the biostructure is a bone repair article for cranioplasty, alveolar ridge augmentation, bone void filler, a spinal fusion or other spinal repair device, or other bone repair article.

32. The biostructure of claim 1, wherein the biostructure is shaped to dimensions derived from medical imaging data.

33. The biostructure of claim 32, wherein the biostructure further includes at least one feature not present in the medical imaging data.

34. The biostructure of claim 1, wherein the biostructure comprises dimensions customized for a particular patient.

35. A biostructure which comprises a matrix which defines pores, wherein at least some pores are partially occupied by an interpenetrant, and wherein some pores are free of the interpenetrant, and wherein the biostructure comprises one or more macroscopic internal features.

36. The biostructure of claim 35, wherein at least some of the macroscopic internal feature(s) are bounded by at least some pores which are substantially free of the interpenetrant.

37. The biostructure of claim 35, wherein the macroscopic internal feature(s) have cross-sectional dimensions of between 100 micrometers and 1000 micrometers.

38. The biostructure of claim 35, wherein the macroscopic internal feature(s) are selected from the group consisting of through channels, dead-ended channels, intersecting channels, straight channels, non-straight channels, constant-cross-section channels and variable-cross-section channels.

39. The biostructure of claim 35, wherein the pores have a total pore volume excluding the internal volume contained within macroscopic internal feature(s), and the interpenetrant has a total interpenetrant volume, and the total interpenetrant volume is less than approximately 80% of the total pore volume.

40. The biostructure of claim 35, wherein the macroscopic internal feature(s) comprise channels each having respective principal directions, the channels intersecting other channels such that at points of intersection, the principal directions of the intersecting channels are substantially mutually perpendicular to each other.

41. The biostructure of claim 35, wherein the macroscopic internal feature(s) comprise channels such that three channels intersect at a common location.

42. The biostructure of claim 35, wherein the biostructure has at least one surface that is not penetrated by macroscopic internal features.

43. The biostructure of claim 35, wherein the biostructure is penetrated by a hole that is suitable for passage of a catheter.

44. A biostructure which comprises a matrix which defines pores, wherein in one region the pores are occupied by an interpenetrant to a greater non-zero extent and in another region the pores are occupied to a lesser non-zero extent.

45. The biostructure of claim 44, wherein the biostructure exhibits a gradient of extent of occupation of the pores by the interpenetrant.

46. The biostructure of claim 44, wherein the biostructure further comprises at least yet another region in which pores are not occupied at all by the interpenetrant.

47. The biostructure of claim 44, wherein the biostructure comprises one or more macroscopic internal features.

48. The biostructure of claim 47, wherein at least some of the macroscopic internal features are bounded by at least some pores that are free of the interpenetrant.

49. The biostructure of claim 44, wherein at least some of the pores that are free of the interpenetrant are on an overall external surface of the biostructure.

50. A biostructure that comprises a matrix that defines pores having a pore size distribution, wherein smaller pores are occupied by an interpenetrant to a greater non-zero extent and larger pores to a lesser non-zero extent, and further comprising macroscopic internal features.

51. The biostructure of claim 50, wherein the biostructure exhibits a gradient of extent of occupation of the non-matrix by the interpenetrant.

52. The biostructure of claim 50, wherein the biostructure further comprises regions in which the pores are not occupied at all by the interpenetrant.

53. A biostructure which comprises a matrix which defines pores, wherein at least some of the pores are partially occupied by an interpenetrant, wherein the interpenetrant has a composition which varies from region to region of the matrix.

54. The biostructure of claim 53, wherein the biostructure comprises one or more macroscopic internal features.

55. A method for forming a biostructure, the method comprising:
fabricating a preform having a matrix which defines pores;
determining a total pore volume within the pores;
calculating a chosen volume of one or more liquid infiltrants, the chosen volume being less than the determined total pore volume;
dispensing onto the preform the chosen amount of the liquid infiltrant(s); and
causing or allowing the liquid infiltrant(s) to harden to form an interpenetrant.

56. The method of claim 55, wherein determining the total pore volume comprises determining the total volume only of pores smaller than a certain size.

57. The method of claim 55, wherein determining the total pore volume comprises excluding the volume of empty space within macroscopic internal features.

58. The method of claim 55, wherein the chosen volume of the liquid infiltrant(s) is less than approximately 80% of the determined total pore volume.

59. The method of claim 55, wherein the dispensing the liquid infiltrant(s) is performed at a single location on the preform.

60. The method of claim 55, wherein the dispensing the liquid infiltrant(s) is performed at multiple locations on the preform.

61. The method of claim 60, wherein the dispensing the liquid infiltrant(s) comprises dispensing specific amounts of the liquid infiltrant(s) at specific locations on the preform.

62. The method of claim 60, wherein the dispensing the liquid infiltrant(s) comprises dispensing different compositions of the liquid infiltrants at specific locations on the preform.

63. The method of claim 62, wherein the different compositions, when hardened, have different resorption characteristics.

64. The method of claim 55, wherein the dispensing the liquid infiltrant(s) is performed by micropipetting.

65. The method of claim 55, wherein the dispensing the liquid infiltrant(s) is performed by automated dispensing means.

66. The method of claim 55, wherein the liquid infiltrant(s) comprises a monomer, and wherein the causing or allowing the liquid infiltrant(s) to harden comprises causing or allowing the monomer to polymerize.

67. The method of claim 55, wherein the liquid infiltrant(s) comprises a monomer together with an activator or initiator or catalyst suitable to cause the monomer to polymerize.

68. The method of claim 55, wherein the liquid infiltrant(s) comprises one or monomers together with one or more polymers, and wherein the causing or allowing the liquid infiltrant to harden comprises causing or allowing the monomer to polymerize.

69. The method of claim 55, wherein the liquid infiltrant(s) comprises a bioactive substance and a polymer.

70. The method of claim 55, wherein the liquid infiltrant(s) comprises a bioactive substance and a monomer.

71. The method of claim 55, wherein the liquid infiltrant(s) comprises a bioactive substance and a monomer and a polymer.

72. The method of claim 55, wherein the liquid infiltrant(s) comprises a substance selected from the group consisting of water-soluble substances, Active Pharmaceutical Ingredients, antibiotics, anti-inflammatories, growth factors, other bioactive substances, chemotherapeutic agents, and anesthetics.

73. The method of claim 55, wherein the causing or allowing the liquid infiltrant(s) to harden comprises heating the biostructure.

74. The method of claim 73, further comprising, prior to the heating, enclosing the biostructure in a sealed bag.

75. The method of claim 55, wherein the dispensing the liquid infiltrant(s) is preformed at a sub-atmospheric pressure.

76. The method of claim 55, wherein the dispensing the liquid infiltrant(s) comprises using suction to direct motion of the liquid infiltrant(s).

77. The method of claim 55, further comprising, before the dispensing the liquid infiltrant(s), treating the preform with a coupling agent.

78. The method of claim 77, wherein the coupling agent is selected from the group consisting of silanes and titanates.

79. The method of claim 55, further comprising, before the dispensing the liquid infiltrant(s), applying a fugitive material to selected locations in the preform.

80. The method of claim 55, wherein the fabricating the preform comprises fabricating by three-dimensional printing.

81. The method of claim 55, wherein the fabricating the preform comprises sintering.

82. The method of claim 55, wherein the fabricating the preform comprises custom designing the preform based on medical imaging data.

83. The method of claim 82, wherein the fabricating the preform comprises adding at least one feature not present in the medical imaging data.

84. The method of claim 55, further comprising, after the hardening, infiltrating yet another substance into space in the biostructure which is not occupied by either the matrix or the interpenetrant.

85. The method of claim 84, wherein the infiltrating is performed only in certain regions of the biostructure.

86. The method of claim 84, wherein the yet another substance is selected from the group consisting of water-soluble substances, Active Pharmaceutical Ingredients, antibiotics, anti-inflammatories, growth factors, other bioactive substances, chemotherapeutic agents, and anesthetics.

87. The method of claim 55, further comprising, at any stage after the fabricating the preform, infiltrating a substance that is selected from the group consisting of water-soluble substances, Active Pharmaceutical Ingredients, antibiotics, anti-inflammatories, growth factors, other bioactive substances, chemotherapeutic agents, and anesthetics.

88. A biostructure made by the method of claim 55.

89. A biostructure comprising a first region having a first circumferential shape and a second region having a second circumferential shape, the first circumferential shape being everywhere larger than the first circumferential shape, the biostructure having channels in it in at least two directions.

90. The biostructure of claim 89, wherein the second circumferential shape is smaller than an opening in a bone of a patient, and the first circumferential shape is larger than the opening in the bone of the patient.

91. The biostructure of claim 89, wherein the biostructure defines pores and at least some pores in at least some regions of the biostructure contain interpenetrant.
